(19) United States
(12) Patent Application Publication
(10) Pub. No.: US 2017/0166619 A1
(43) Pub. Date: Jun. 15, 2017

(54) Polypeptide connected with an organic residue

(71) Applicant: Morphoplant GmbH, Bochum (DE)

(72) Inventor: Herbert P. Jennissen, Essen (DE)

(21) Appl. No.: 15/530,271

(22) Filed: Dec. 16, 2016

Related U.S. Application Data
(63) Continuation of application No. 11/631,231, filed on Dec. 12, 2007, now abandoned.

(30) Foreign Application Priority Data
Jun. 29, 2004 (DE) 10 2004 031 258.3

(51) Int. Cl. C07K 14/51

(52) U.S. Cl. CPC C07K 14/51; A61K 47/48038

(57) ABSTRACT

The invention relates to a method for producing a polypeptide which is modified with an organic group, wherein a bioactive polypeptide is covalently bound to an organic group that comprises a backbone structure having aromatic side chains, thereby forming a modified polypeptide which is constituted of the bioactive polypeptide and the group having aromatic side chains. At least one of the aromatic side chains of the group is subjected to chemical or enzymatic hydroxylation.

The invention refers to a method of producing of a polypeptide which has been modified with an organic residue, the modified polypeptides thus produced and the use thereof.

Amino acid sequence motifs at the end of a protein hybrid are also referred to as “tag” (English for label, labelling, molecule group which is active upon binding). Previous concepts for the production of such protein-tags normally emanate from general affinity reactions (enzyme-substrate, effector-receptor, biotin-avidine or antigene-antibody reactions) and make use of the high affinities of one partner which is bound to a surface to bind the second partner. An especially common method is the his-tag method in which a polyhistidine tail on a fusion protein serves to form a chelate complex with immobilized metal ions, such as Zn²⁺, Cu²⁺, Ni²⁺ (=metal chelate affinity technology) on a surface. Other concepts and techniques for binding of molecules, like proteins on metal surfaces, are based on, e.g., fixing a nucleophilic group on a metal surface by means of a silanization reaction, which is then reacted in a second reaction with the protein, wherein both molecules are linked via a covalent bond.

Disadvantages of the silanization process can be seen in the fact that these chemical reactions need to be carried out in anhydrous environment and are thus laborious and expensive. Furthermore, these radical reactions often lead to denaturation of the proteins immobilized.

A great disadvantage of the tag technique described above is that not only the protein needs to be modified genetically by attaching, for example, his-tags but also the surface binding the his-tag need to be modified with organic chelating molecules which carry the immobilized Zn²⁺, Cu²⁺ or Ni²⁺-ions to be able to react with the poly-histidine residues.

A further great disadvantage of the method described is that the surface carrying the ions is instable because of the relative low affinity of the chelating group to the Zn²⁺, Cu²⁺, Ni²⁺ ions immobilized and can thus only be used for a short time to bind the his-tag protein.

The problem to be solved by the present invention can thus be regarded as conferring high affinity to peptides compared to metal surfaces, glass or ceramics without the need of modifying such surfaces in advance.

According to the invention the problem described above can be solved by providing a method for producing a polypeptide modified with an organic residue in which a bioactive polypeptide with an organic residue comprising a backbone structure with aromatic side chains is bound covalently, and thus a modified polypeptide made of a bioactive polypeptide and an organic residue with aromatic side chains is formed and at least one aromatic side chain of the organic residue is hydroxylated chemically or enzymatically.

Residues made of separate monomers which can be the same or different and which have aromatic side chains which are directly adjacent or separated by one or more monomers are usable as organic residues of the present invention. Examples for such organic residues are such with backbone structure (backbone) having C atoms, like polymers selected from ethylene, propylene, amides, ester-, ether- or thioester compounds which have aromatic side chains, such as phenyl- or naphthylgroups, heterocycles, aromatic amino acids, like Phe, Tyr or Trp etc and which carry in addition at least one hydroxyl group at the organic residue. Exemplarily, at least partly hydroxylated polystyrene or amino acid sequences having aromatic hydroxyl substituted amino acids are mentioned. The number of monomers can be up to about 50 wherein at least more than about 5 hydroxyl groups should be present in the organic residue to provide for a sufficient interaction with the surface.

As a result of the hydroxyl groups present at the aromatic side chain, the interactions between the organic residue as tag and the surface of the material have a substantially higher stability than, for example, a his-tag, so that a long term coating is possible. The spectrum of possible uses of such an organic tag technique constitutes an enormous enhancement in the field of tissue engineering and biomaterial technologies.

In this way, it is possible to link the polypeptide as previously described via the organic residue on the substrate surface and thus make it usable for further applications, wherein the polypeptide comprises preferably growth factors of the TGF-β-superfamily, such as TGF-β1 or bone growth factors, such as BMP-2, BMP-7, cartilage building factors, such as CDMP (GDF-5), or blood vessel growth factors, such as VEGF or angiotropine, as well as PDGF and Nell-proteins, such as nell-1 and nell-2, as possible bioactive mediators, factors or tissue hormones.

Thereby, the organic residue can be bound at the N-terminal or C-terminal of the polypeptide, or the organic residue having aromatic side chains can be interposed into the polypeptide as long as the activity of the polypeptide is not affected adversely.

At the same time it is especially preferred that at least one aromatic side chain of the organic residue is hydroxylated chemically or enzymatically so that this aromatic side chain carries two hydroxyl groups. In this way, an interaction with the surface of a substrate having an especially good quality can be achieved, in particular with such substrates carrying an oxy or hydroxy group, which have a surface made out of metal oxide, metal hydroxide, calcium hydroxyphosphonate (hydroxyapatite), silicium oxide or -hydroxide as it can be found with metals, ceramics or glasses.

In another embodiment of the method, in a first step a bioactive polypeptide is covalently bound to an organic residue which comprises a backbone structure with aromatic side chains, and a modified polypeptide made of a bioactive polypeptide and an organic residue having aromatic side chains is formed, having the following structure:

P—[R₁—(X)ₙ]_{w}—[R₂—(X)ₙ]_{z}—R₃
wherein:
P represents the bioactive polypeptide which is linked C-terminal or N-terminal with the organic residue having aromatic side chains or in which the organic residue having aromatic side chains is interposed;
R₁, R₂ and R₃ are the same or different and each represents an aromatic amino acid which is selected from the group consisting of tyrosine, tryptophane or phenylalanine;
X represents any amino acid which is the same or different within the units [R₁—(X)ₙ]_{w} and [R₂—(X)ₙ]_{z};
n is 0 to 10 inclusively;
w and z represent a natural number from about 0 to 50; and
in a second step at least one of R₁, R₂ and R₃ is modified chemically or enzymatically in such a way that at least two hydroxyl groups are present at the aromatic ring.

The natural aromatic amino acids are L-phenylalanine (Phe), L-tyrosine (Tyr) and L-tryptophane (Trp). Since in the meantime also the existence of D amino acids in mammals is proven, also D-phenylalanine, D-tyrosine and D-tryptophane might be used for such residues. Genetically producible organic amino acid sequences which can be used according to the present invention might consist of n=2-50 aromatic amino acid sequences, like -(Phe)ₙ-, -(Tyr)ₙ-, -(Trp)ₙ- or combinations thereof, which are bound N-terminal or C-terminal at the target-polypeptide. For X, these amino acid sequences can comprise any amino acid which is the same or different within the units [R₁—(X)ₙ]_{w} and [R₂—(X)ₙ]_{z}. Thereby, also such analog compounds are included in which the stereo chemistry of the separate amino acids is changed in one or more specific positions from L/S to D/R. Also included are analog compounds which possess a peptide character only to a lesser extent. Such peptide mimetics can comprise for example one or more of the groups of the following substitutions for CO—NH-amid linkages: depsipeptide (CO—O), iminomethylene (CH₂—NH), trans-alkene (CH═CH), enaminonitrile (C(═CH—CN)—NH), thioamide (CS—NH), thiomethylene (S—CH₂), methylene (CH₂—CH₂) and retro-amide (NH—CO) which, for example, increase the stability of the organic residue P—[R₁—(X)ₙ]_{w}—[R₂—(X)ₙ]_{z}—R₃ compared to proteases in a physiological environment. These substitutions can be used within the organic residue of the invention at every spot where peptide linkages can be found.

Hydroxylation of the aromatic side chains can be performed by known chemical procedures or enzymatically. Therefore, in the method of the present invention it is preferred that at least one aromatic residue is hydroxylated chemically or enzymatically so that two hydroxyl groups are present adjacent at the aromatic ring, more preferably three hydroxyl groups are present adjacent at the aromatic ring.

In this way, a chemical approach can be followed for the synthesis of the organic residue and this organic residue can be bound to an amino acid residue of the bioactive polypeptide. Alternatively, a polypeptide modified with an organic residue can be genetically synthesized in pro- or eukaryotic cells.

In one embodiment of the inventive method n is smaller than three, preferably equal 0 or 1, and w and z are each an integral number from about 1 to 5 in the above formula P—[R₁—(X)ₙ]_{w}—[R₂—(X)ₙ]_{z}—R₃.

Thus, the present invention further refers to a polypeptide modified with an organic residue, which is formed out of a bioactive polypeptide and an organic residue having aromatic side chains, wherein at least one aromatic side chain of the organic residue is hydroxylated chemically or enzymatically.

In this way, the organic residue of the peptide has preferably two hydroxyl groups in at least one aromatic side chain.

In addition, the invention refers also to a polypeptide having the following structure:

P—[R₁—(X)ₙ]_{w}—[R₂—(X)ₙ]_{z}—R₃

wherein
P represents the bioactive polypeptide which is linked C-terminal or N-terminal with the residue having aromatic side chains or in which the organic residue having aromatic side chains is interposed;
R₁, R₂ and R₃ are the same or different and each represents an aromatic amino acid which is selected from the group consisting of tyrosine, tryptophane or phenylalanine;
X represents any amino acid which is the same or different within the units [R₁—(X)ₙ]_{w} and [R₂—(X)ₙ]_{z};
n is 0 to 10 inclusively;
w and z represent a natural number from about 0 to 50;
wherein at least one of R₁, R₂ and R₃ is modified chemically or enzymatically in such a way that at least two hydroxyl groups are present at the aromatic ring.

Of particular importance as organic residues are poly-phe, poly-tyr and poly-trp, which can interact for example on a metallic surface directly via π-π or d-π donor-acceptor interactions with corresponding π- or d-electron containing compounds due to their aromatic character. Furthermore, they can be converted in corresponding hydroxy compounds after introducing hydroxyl groups by means of oxidation processes. For example, tyrosine is a natural aromatic hydroxy compound. For example, by introducing another hydroxyl group in tyrosine dihydroxyphenylalanine is formed and out of a corresponding poly-tyr (-(tyr)ₙ-) a poly-DOPA (-(DOPA)ₙ-) is formed.

Using a method of coating a substrate allows applying a solution of a polypeptide on the surface of a substrate and immobilizing the polypeptide via covalent or non-covalent interaction on the surface of the substrate. In particular, the substrate can be made of metal, ceramic or glass and have a surface made of metal oxide, metal hydroxyide, calcium hydroxyphosphonate (hydroxyapatite), silicium oxide or -hydroxide carrying oxy- or hydroxy groups.

In case of poly-tyr, the poly-tyrosine-tag is already present as polyphenolic group after the first step and can be used directly for a binding reaction, e.g., on metal surfaces. However, it can be expected that introducing of a second phenolic hydroxyl group in tyrosine leads to an increase of binding specifity and affinity (=binding energy). Thus, in a second step one or more phenolic hydroxyl groups can be introduced into the aromatic ring system of phenylalanine, tyrosine or tryptophane. Following this way, the amino acid tyrosine (4-hydroxy-phenylalanine) can be transferred, for example, to 3,4-dihydroxyphenylalanine (DOPA). Thus, a poly-DOPA-tag can be produced out of a poly-tyrosine-tag. A further hydroxylation to 3,4,5-trihydroxyphenylalanine (TOPA) is also possible. The polyphenolic tag, e.g. poly-DOPA-tag, can then confer to proteins specific adhesion properties on metal surfaces, in particular transition metals, glass surfaces or ceramics, so that a permanent coating of the surface material can be provided for varied biological, chemical and medical applications.

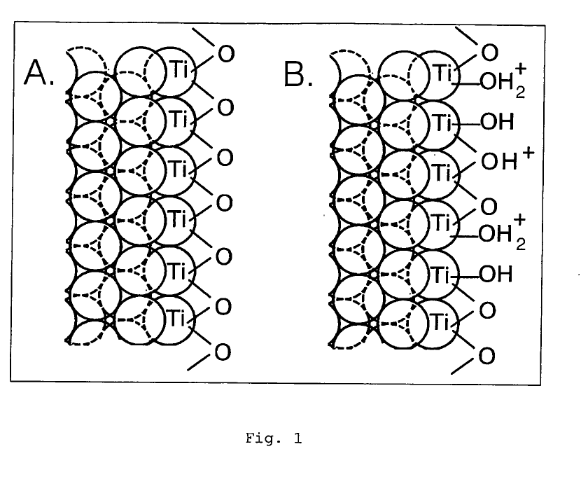

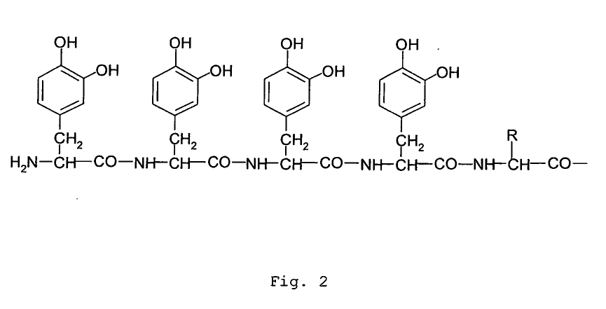

Polyphenolic tags, such as poly-DOPA, can undergo specific binding reactions with certain transition metal oxides on metal surfaces. The following chemical reaction types for binding of a protein via a poly-DOPA-tag to a titanium surface are possible:
1. Ionic interactions between positive charges on the titanium surface (FIG. 1B) and negative charged phenolate ions of poly-DOPA (FIG. 2).
2. Electron-donor-acceptor complex in the form of a d-n interaction between titanium (d-orbital) and the n-electrons of the phenolic ring.
3. It might also be possible that a direct metalorganic linkage between DOPA and the titanium surface is assembled.

The specific adhesion properties, for example of poly-DOPA-tags to fusion proteins are used in the method of the present invention to directly immobilize proteins selectively and with high affinity on metal- or glass surfaces. Presumably, the hydroxyl groups in ortho position at the phenyl residue (i.e. DOPA) of the (DOPA)₃ structure are responsible for the high affinity binding of residual-DOPA at the hydroxyl groups of a titanium dioxide surface which can be found on metallic titanium as it is shown in FIG. 1. Thus, possibility 3 (supra) was fully verified. However, possibilities 1 and 2 are thus not excluded but can act additionally.

By simultaneous reaction of several DOPA-residues in a poly-DOPA molecule with the titanium surface, the affinity of the bond will increase in a power function so that extremely high binding affinities (10⁸-10¹⁵ M⁻¹) can be reached. Transition metal oxide containing surfaces can be transformed to support materials for proteins carrying organic residues and can be used for synthesis of biological active surfaces in the area of tissue engineering and biomaterial engineering. Application of this technology is also possible on glass surfaces. Thus, matrices for natural, recombinant or synthetic proteins or peptides can be prepared, which can also prove of value in the area of chromatography, immunoassays and array technology.

Possible bioactive peptides, like mediators, factors or tissue hormones that can be used are preferably growth factors of the TGF-β-superfamily, like TGF-β1, or bone growth factors, like BMP-2, BMP-7, cartilage forming factors, like CDMP (GDF-5), or blood vessel growth factors, like VEGF or angiotropine as well as PDGF and Nell-proteins, such as nell-1 and nell-2.

The single or multiple hydroxylated aromatic polyamino acids which are covalently bound with a distinct target protein, like an enzyme, growth factor (supra) or a structural protein serve as anchor structure for the tight linkage of the target-protein to a silicium oxide- or metal oxide containing matrix. Through this bond to the oxide containing matrix, the fusion protein can be purified from an extract, or can be immobilized in a biological active form on a metal surface, for example of a titanium implant.

Synthesis of a polyphenolic tag will be described in more detail by using the example of poly-L-tyrosine and poly-L-DOPA. Analog methods can be prepared for polyphenylalanine- and polytryptophane-tags. A method will be described which can be carried out in an aqueous environment and under gentle conditions. The fusion protein desired in which the N-terminus or C-terminus must exist free, i.e. not hidden within the proteins, will be manufactured as described in the following three steps:

The triplet codes for tyrosin are UAU and UAC. Consequently, (UAU)ₙ and (UAC)ₙ which are fused with a target protein at the C-terminus will result in a protein-(tyr)n (precursor polypeptide):

cDNA-(UAC)ₙ→protein-C-term-(tyr)ₙ (1)

(UAC)n-cDNA→(tyr)ₙ-N-term-protein (2)

The number of tyrosine residues is preferably between about 3-5, wherein the tyrosine residues follow in succession, like in formula 1 and 2 or are separated by other amino acids (heteropolymer), for example in the formula P-[tyr-(X)ₙ]_{w}-[tyr-(X)ₙ]_{z}-tyr, wherein X can be any amino acid in any sequence and n is preferably between about 1 to 5. Because tyrosine and polytyrosine are poorly soluble in water, acidic or basic amino acids for X in the formula P-[tyr-(X)ₙ]_{w}-[tyr-(X)ₙ]_{z}-tyr are preferred if the solubility of the fusion proteins shall not be lowered due to the poly-tyr-tag. It might be of particular advantage to incorporate tyrosine in certain defined distances within the polypeptide, so that the hydroxyl groups can adapt to the surface topography of the hydroxyl groups of the metal oxides. The protein thus produced genetically in, for example, E. coli or in CHO-cells (chinese hamster ovary cells) needs than to be enriched and purified. Therefore, a double tag can be used for purification which consists of a N-terminal poly-tyr-tag and a C-terminal poly-his-tag. Alternatively, it could be possible to combine the poly-tyr-tag with a poly-his-tag and at some point thereafter to cleave the poly-his-tag by known methods. It is also possible to purify the protein, e.g. rhBMP-2, using classic methods.

In the next step, the aromatic amino acid phe, tyr, DOPA is hydroxylated. In case of tyrosin this can be done chemically or enzymatically.

Peptidyl tyrosine hydroxylase or mushroom tyrosinase (tyrosinase) together with oxygen and a reducing agent NADH+H⁺ or ascorbic acid results in

protein-(tyr)ₙ→protein-C-term-(DOPA)ₙ (3)

Conversely, (UAU)ₙ and (UAC)ₙ in fusion with a target protein at the N-terminus under similar conditions results in:

(tyr)ₙ-N-term-protein→protein-C-term-(DOPA)ₙ (4)

Furthermore, it is also possible herein, as previously described above, that homo- or heteropolymers of the precursor tyrosine peptides are present which can be transformed into the corresponding homo- or heteropolymers of DOPA afterwards. In this case it could be of particular importance that the DOPA molecules maintain a defined distance within the polypeptide which corresponds to the specific steric proportion of the metal oxide layer of the metal surface. In a similar way, polyanorganic amino acid hybrids can be prepared based on the amino acids phenylalanine and tryptophane.

According to a third possibility, [R₁—(X)ₙ]_{w}—[R₂—(X)ₙ]_{z}—R₃, such as poly-X sequences, can also be interposed in a protein as long as the biological activity allows it. One can imagine such an application for the case that the N-terminus or the C-terminus are not allowed to be modified. One can imagine such an application for the case that the N-terminus or the C-terminus are not allowed to be modified due to reasons of activity or in case the terminus is not free but is located on the inside of the protein.

The fusion proteins produced in the methods just described can than be bound to the metal-, ceramic- or glass surfaces via the polyorganic amino acid hybride. In a particular embodiment with BMP-2 (bone morphogenetic protein 2) described herein as an example, a poly-DOPA- or poly-TOPA-tag fused at the N-terminus can be used to bind BMP-2 in biological active form with high affinity to the titanium surface simply by incubating it with a titanium implant, and to use it as bioactive tooth-, hip- or knee implant for humans. A high bioactivity of BMP-2 can be expected because N-terminal peptide extensions, similar to poly-DOPA-tags with 5-10 amino acids, normally do not lead to a loss of biological activity. Furthermore, binding of BMP-2 via a N-terminal poly-DOPA-tag leads to an immobilization of BMP-2 on the titanium surface in a specific orientation and thus brings about an excellent high specific biological activity. Alternatively, proteins with a poly-DOPA-tag, for example, could be bound to glass micro beads or silica beads, and could be used as affinity ligands in affinity chromatography.

The invention will be described in more detail based on the following figures and examples. Thereby, it is shown in

FIG. 1 the structure model of the titanium dioxide surface of a titanium material as model for an oxide layer of transition metals or a glass surface; and

FIG. 2 the structure of poly-3,4-dihydroxyphenylalanine-tags (=poly-DOPA-tag) in a N-terminal location at a hypothetical fusion protein, for example of the TGF-β family.

As shown in FIG. 1, partial cutout A and B show a non-hydrolyzed oxide layer (A) and a hydrolized oxide layer with protonated groups (B). The isoelectric point is about pH 4.5. The kind of reactions of the hydroxyl groups of the hydrolyzed oxide layer is the following:

Terminal Hydroxyl Group:

≡TiOH₂⁺—H⁺⇄≡TiOH—H⁺⇄≡TiO⁻ (1)

Hydroxyl Bridge Group:

≡Ti—OH⁺—Ti≡—H⁺⇄≡Ti—O—Ti≡ (2)

As shown in FIG. 2, the structure of a poly-3,4-dihydroxyphenylalanine-tag (=poly-DOPA-tag) is specified which is localized N-terminal at a hypothetical polypeptide, which is not shown, for example of the TGF-β family. Thereby, the phenolate groups of the poly-DOPA can dissociate into a proton and the corresponding negatively charged phenolate ion (pK ˜10.0).

### EXAMPLES OF PRODUCTION

### Example 1—Hydroxylation of Tyrosine Containing Peptides

### Phosphate-Borate-Ascorbate Buffer:

0.1 M phosphate buffer
0.02 M borate

Adjusting the pH-value with ascorbic acid to pH 7.0

### Preparative Approach for Model Peptides:

10 ml phosphate-borate-ascorbate buffer, pH 7.0
2 mg mushroom tyrosinase (Sigma, 6680 U/mg)
10 mg tyrosine peptide (tyr-tyr-lys-his-lys-tyr-tyr or ala-lys-pro-ser-tyr-pro-pro-thr-tyr-lys)

Incubation for 40 minutes (20-30° C.) under stirring (or sparging with oxygen).

Yield of DOPA containing peptide: ˜80%

### Example 2—Hydroxylation of rhBMP-2 with an N-Terminal Poly-Tyr-Tag (3-5 tyr)

For hydroxylation of rhBMP-2 one must use another buffer system as in example 1 for the artificial peptides because rhBMP-2 is poorly soluble at pH 7.0.

### Borate Buffer:

0.125 Na-borate, pH9-10
0.066% sodium dodecyl sulfate
25 mM ascorbic acid

### Preparative Approach for BMP-2:

10 ml borate-ascorbate buffer, pH 9.10
2 mg mushroom tyrosinase (Sigma, 6680 U/mg)
2 mg rhBMP-2 with poly-tyr-tag (n=3-5)

Incubation for 30-40 minutes (20-30° C.) under stirring (or sparging with oxygen).

The hydroxylation reaction takes place much faster at a pH of 9.0 than at a pH of 7.0. In the present case, this is an advantage because rhBMP-2 is nearly insoluble at a pH of 7.0 but is extremely good dissolvable at a pH of 9-10 in the borate buffer indicated above.

Even though it can be expected that hydroxylation of the poly-tyr-tags at the poly-tyr-rhBMP-2 will also hydroxylate tyrosine residues in the molecule network of BMP-2 itself, this does not have any effect on the biological activity of rhBMP-2 according to the inventor. For instance, iodization experiments, wherein tyrosine residues in rhBMP-2 molecules have been iodized with ¹²⁵I according to the chloramin T method, have shown, however, that the biological activity of rhBMP-2 is fully maintained. Thus, it can be concluded that a modification of the tyrosine residues by incorporation of a second hydroxyl group will also not lead to an impairment of the biological activity. It can be concluded, that presumably no tyrosine residues are directly involved in the biological activity of rhBMP-2.

In case of BMP-2 also the modification of the N-terminal end will not lead to an activity loss of BMP-2. It was already shown, that the 12 N-terminal amino acids of rhBMP-2 can be replaced by a foreign peptide with 17 amino acids (i.e. it is 5 amino acids longer!) without decreasing the biological activity. Therefore, it should be possible without any problems to fuse genetically a penta-peptide having 3-5 tyrosine residues to the N-terminus of rhBMP-2 without impairing the activity. It was also possible based on the X-ray structure of rhBMP-2 to show that the N-terminus of rhBMP-2 is present free to move. That is so to say, the N-terminus cannot be displayed in the X-ray analysis because of its free movability.

1. A polypeptide modified with an organic residue, characterized in that
the modified polypeptide which is composed of a bioactive polypeptide which is bound covalently with an organic residue comprises a backbone structure having aromatic side chains,
wherein the organic residue comprises a backbone structure having aromatic side chains selected from the group consisting of
(i) polymers with up to about 50 monomers of ethylene, propylene, ester-, ether- or thioether compounds having side chains which are phenyl- or naphtyl groups, heterocycles or aromatic amino acid, like phe, tyr or trp, which carry at least one hydroxyl group at the aromatic residue, and wherein at least one aromatic side chain of the organic residue is hydroxylated chemically or enzymatically in such a way that this aromatic side chain carries two hydroxyl groups;
(ii) a residue with the structure
P—[R₁—(X)ₙ]_{w}-[R₂—(X)ₙ]_{z}—R₃
wherein
P represents the bioactive polypeptide which is connected C-terminal or N-terminal with the organic residue having aromatic side chains or in which the organic residue having aromatic side chains is interposed;
R₁, R₂ and R₃ are the same or different and each represents an aromatic amino acid which is selected from the group consisting of tyrosine, tryptophane or phenylalanine;
X represents any amino acid which is the same or different within the units [R₁—(X)ₙ]_{w} and [R₂—(X)ₙ]_{z};
n is 0 to 1;
w and z represent a natural number from about 0 to 5;
and wherein at least one of R₁, R₂ and R₃ is hydroxylated chemically or enzymatically in such a way that at least two hydroxyl groups are present at the aromatic ring;
(iii) the amino acid sequences -(phe)ₙ-, -(tyr)ₙ- or -(trp)ₙ- with n=2-50 or combinations thereof, wherein at least one phe, tyr or trp is hydroxylated chemically or enzymatically in such a way that at least two hydroxyl groups are present at the aromatic ring; and
(iv) analog compounds of the peptides listed under (ii) and (iii), wherein the CO—NH-amid linkages are substituted by one or more of the groups consisting of depsipeptide (CO—O), iminomethylene (CH₂—NH), trans-alkene (CH═CH), enaminonitrile (C(═CH—CN)—NH), thioamide (CS—NH), thiomethylene (S—CH₂), methylene (CH₂—CH₂) and retro-amide (NH—CO).

2. The polypeptide according to claim 1, wherein at least one aromatic side chain of the organic residue is hydroxylated chemically or enzymatically in such a way that two hydroxyl groups are present adjacent at the aromatic ring.

3. The polypeptide according to claim 1, wherein at least one aromatic side chain of the organic residue is hydroxylated chemically or enzymatically in such a way that three hydroxyl groups are present adjacent at the aromatic ring.

4. The polypeptide according to claim 1, wherein the organic residue has the structure P—[R₁—(X)ₙ]_{z}—[R₂—(X)ₙ]_{z}—R₃, wherein P represents the bioactive polypeptide which is connected C-terminal or N-terminal with the organic residue having aromatic side chains or in which the organic residue having aromatic side chains is interposed; R₁, R₂ and R₃ are the same or different and each represents an aromatic amino acid which is selected from the group consisting of tyrosine, tryptophane or phenylalanine; at least one of R₁, R₂ and R₃ is tyrosine; X represents any amino acid which is the same or different within the units [R₁—(X)ₙ]_{w} and [R₂—(X)ₙ]_{z}; n is 0 to 1; w and z represent a natural number from about 0 to 5; and wherein at least one of R₁, R₂ and R₃ is hydroxylated chemically or enzymatically in such a way that at least two hydroxyl groups are present at the aromatic ring.

5. The polypeptide according to claim 4, wherein at least two 3,4-dihydroxyphenylalanine(DOPA)-residues are formed by chemical or enzymatic hydroxylation.

6. The polypeptide according to claim 1, wherein the organic residue, which has aromatic side chains, has the amino acid sequence -(phe)ₙ-, -(tyr)ₙ- or -(trp)ₙ- with n=2-50, which are linked to the bioactive polypeptide C-terminal or N-terminal in form of a fusion protein or are interposed in the bioactive polypeptide, wherein at least one of phe, tyr or trp is hydroxylated chemically or enzymatically in such a way that at least two hydroxyl groups are present at the aromatic ring.

7. The polypeptide according to claim 6, wherein the organic residue is fused to the bioactive polypeptide at the C- or N-terminus.

8. The polypeptide according to any of claims 6 to 7, wherein the organic residue having aromatic side chains is -(tyr)ₙ- with n=2-50, wherein -(tyr)ₙ- is hydroxylated chemically or enzymatically in such a way that -(DOPA)ₙ- is formed.

9. The polypeptide according to claim 6, wherein n is two to five.

10. The polypeptide according to claim 1, wherein the bioactive polypeptide is a growth factor.

11. The polypeptide according to claim 10, wherein the growth factor is selected from the group consisting of growth factors of the TGF-β superfamily, bone growth factors of the BMP family, cartilage forming factors, blood vessel growth factors and nell proteins.

12. The polypeptide according to claim 1, wherein the synthesis of the organic residue has been carried out chemically and this organic residue is bound covalently to an amino acid residue of a bioactive polypeptide.

13. The polypeptide according to claim 1, wherein the polypeptide modified with an organic residue is synthesized genetically in pro- or eukaryotic cells.

14. A method for coating a substrate, wherein a solution of the polypeptide according to claim 1 is applied to a surface of a substrate and the polypeptide is immobilized on the surface of the substrate by covalent or non-covalent interactions.

15. The method according to claim 14, wherein the substrate is metal, ceramic or glass.

16. The method according to claim 15, wherein the substrate has a surface carrying oxy- or hydroxy groups and is made of metal oxide, metal hydroxide, calcium hydroxyphosphonates (hydroxyapatite), siliciumoxide or -hydroxid.

17. The method according to claim 14, 15 or 16, wherein the substrate is implanted in form of an implant for animals or human.

18. The method according to claim 17, wherein the polypeptide is implanted for coating the implant in form of a growth factor BMP-2 or BMP-7 linked to a poly-DOPA- or poly-TOPA-tag.

19. An implant obtainable by the method according to claim 14.

20. Use of a polypeptide according to claim 1 in an analytical method for detecting of immunoglobulin or cell receptors.

21. Use of the polypeptide according to claim 1 in an affinity chromatography method.
